(19) United States
(12) Patent Application Publication
(10) Pub. No.: US 2024/0325633 A1
(43) Pub. Date: Oct. 3, 2024

(54) INTRAVASCULAR ARTIFICIAL PANCREAS

(71) Applicant: IVIVA Medical, Inc., Woburn, MA (US)

(72) Inventor: Daniel Bowers, Reading, MA (US)

(21) Appl. No.: 18/434,734

(22) Filed: Feb. 6, 2024

Related U.S. Application Data
(60) Provisional application No. 63/483,316, filed on Feb. 6, 2023.

(51) Int. Cl. A61M 5/142; A61M 5/168; C08L 67/04; C12N 5/071

(52) U.S. Cl. CPC A61M 5/14276; A61M 5/16836; C08L 67/04; C12N 5/0677; A61M 2205/0244; A61M 2205/04

(57) ABSTRACT

Disclosed herein are intravascular artificial pancreas devices and methods for their manufacture.

### RELATED APPLICATION(S)

This application claims the benefit of U.S. Provisional Application No. 63/483,316 filed on Feb. 6, 2023. The entire teachings of the above application are incorporated herein by reference.

### BACKGROUND OF THE INVENTION

The discovery of insulin 100 years ago made insulin dependent diabetes mellitus (IDDM, or Type 1 Diabetes, T1D) a chronic disease now affecting at least 1.6 million Americans. Still, daily management of the disease leaves the majority of patients failing to achieve glycemic control targets. Poor glucose control is linked to the onset of complications including vascular damage that manifests as blindness, kidney failure, amputations and others. A cellular solution for insulin replacement could provide tight glucose control without patient input.[1] Pancreas transplantation can restore glucose control in diabetic patients at least for a period of time, however immunosuppression and an insufficient supply of donor organs limits this treatment. More than 110,000 people in the US alone are waiting for an organ of any type.[2] Bioartificial organs can have a significant effect on the organ waiting list by multiplying the number of patients one organ can treat and acting as a platform for methods to circumvent the need for immunosuppression.

An attractive alternative to pancreas transplant that requires less tissue volume and is adaptable to various implant procedures is pancreatic islet transplantation. The pancreatic islets are the endocrine portion of the pancreas that constitutes ˜10% of pancreas mass. Pancreatic islet transplantation into the portal circulation has resulted in reduction of required exogenous insulin and prevention of severe hypoglycemic events in clinical trials[3,4]. However, complete insulin independence has proven difficult to achieve. Identified factors that contribute to reduced function include: the instant blood mediated immune reaction (IBMIR), allo- and auto-immune reactions, lack of revascularization,[5] immune suppression drug toxicity, and hypoxia at the core of the cell cluster.[6] Promising methods to address these issues include macroencapsulation, microencapsulation, nanoencapsulation, 3D printed scaffolds, and others that have been extensively reviewed and investigated. [7-10][11-13] Careful selection of materials and additives can increase vascular growth and the partial pressure of oxygen,[14] decrease inflammation, make the cells retrievable, and provide a platform to monitor the graft.[15,16] Islet blood flow is one of the highest in the human body. Islet blood flow approximates 5-6 mL/min/g islet mass[17], corresponding to an estimated arteriolar blood flow per islet of 10-20 nL/min. Clinically used sites of the liver (˜1 mL/min/g) and subcutaneous fat (˜0.03 mL/min/g) are less perfused than native islets (FIG. 1), demonstrating the need for an engineered vasculature that can provide the required flow. Several approaches have been examined to compensate for the lower blood flow, such as oxygen supply and microvascular induction.[18,19] Described herein are engineered vasculatures designed to provide the required blood flow immediately after surgical implantation and anastomosis to enable superior graft survival and function.

### SUMMARY OF THE INVENTION

In some aspects, the invention is directed to an intravascular artificial pancreas device capable of producing insulin comprising: a first vascular layer comprising a plurality of first vascular channels each having a first end and a second end, wherein each of the first ends of the plurality of vascular channels connect to a first input conduit and each of the second ends of the plurality of vascular channels connect to a first output conduit, thereby forming a first vascular channel network; an islet layer comprising pancreatic islet and/or beta cells disposed within at least one islet chamber, wherein the pancreatic islet and/or beta cells are further embedded within an islet chamber matrix; and a first thin electrospun membrane disposed as a biomolecule and gas permeable interface between the plurality of vascular channels of the first vascular layer and a first side of the at least one islet chamber, wherein the plurality of vascular channels and the first side of the at least one islet chamber are juxtaposed from each other across the first thin electrospun membrane, thereby permitting exchange of biomolecules and gases between the pancreatic islet and/or beta cells disposed within the at least one islet chamber and the plurality of vascular channels in the first vascular layer.

In some embodiments, the at least one islet chamber comprises a plurality of islet chambers configured as a plurality of islet channels, and at least two vascular channels of the plurality of vascular channels interface with and are juxtaposed across from a first side of each islet channel through a first thin electrospun membrane.

In some embodiments, the intravascular artificial pancreas device as disclosed herein, further comprising: a second vascular layer comprising a plurality of vascular channels, each having a first end and a second end, wherein each of the first ends of the plurality of vascular channels connect to a second input conduit and each of the second ends of the plurality of vascular channels connect to a second output conduit, thereby forming a second vascular channel network; and a second thin electrospun membrane disposed as a biomolecule and gas permeable interface between the plurality of vascular channels of the second vascular layer and a second side of the at least one islet chamber, thereby permitting exchange of biomolecules and gases between the pancreatic islet and/or beta cells disposed within the at least one islet chamber and the plurality of vascular channels in the second vascular layer.

In some embodiments, the at least one islet chamber comprises a plurality of islet chambers configured as a plurality of islet channels, and wherein at least two vascular channels of the plurality of vascular channels interface with and are juxtaposed across from a second side of each islet channel through a second thin electrospun membrane.

In some embodiments, the plurality of vascular channels of the first vascular layer and/or the second vascular layer are lined with endothelial cells. In some embodiments, the endothelial cells are glomerular microvascular endothelial cells or human umbilical vein endothelial cells. In some embodiments, the beta cells are hypo-immune (B2M−/−, CIITA−/−) and/or derived from induced pluripotent stem cells (iPSC).

In some embodiments, the intravascular artificial pancreas device is glucose responsive, producing an amount of insulin in proportion to an amount of glucose within the device.

In some embodiments, the plurality of vascular channels of the first vascular and/or the second vascular layer are microchannels that form a first and/or a second microvascular network.

In some embodiments, the at least one islet chamber comprises elongated first and/or second sides to permit increased surface area for interfacing with one or more vascular channels across the first and/or second thin electrospun membrane.

In some embodiments, the amount of islets and/or beta cells present in the device comprises at least 500,000 islet equivalents. In some embodiments, the at least one islet chamber is capable of accommodating approximately 660,000 islet equivalents.

In some embodiments, the islet chamber matrix comprises collagen. In some embodiments, the islet chamber matrix comprises an in-situ polymerized matrix.

In some embodiments, the thin electrospun membrane comprises polycaprolactone and gelatin. In some embodiments, the first and/or second side of the at least one islet chamber interface with the plurality of vascular channels of the first and/or second vascular layer through the first and/or second thin electrospun membrane at a distance of approximately 10-30 micrometers (μm).

In some embodiments, the islet layer and/or the first and/or second vascular layers further comprise an encasement matrix which encases the first and/or second vascular channel network and the at least one islet chamber.

In some embodiments, the first and/or second inlet conduits and/or outlet conduits are tapered, to minimize shear stress applied across the first and/or second vascular networks. In some embodiments, the first and second inlet conduits are fluidly connected to a first end of a fluid supply conduit and the first and second outlet conduits are fluidly connected to a first end of a fluid exit conduit. In some embodiments, the fluid supply conduit and fluid exit conduit comprise second ends which are proximate to each other and/or are on the same side of the device.

In some embodiments, the first vascular layer, the islet layer, the first thin electrospun membrane and optionally the second vascular layer and second thin electrospun membrane form a first device unit, and wherein the intravascular artificial pancreas device further comprises a second device unit comprising a first vascular layer, an islet layer, a thin electrospun membrane and optionally a second vascular layer and second thin electrospun membrane, that are identical to the first vascular layer, the islet layer, the first thin electrospun membrane and the optional second vascular layer and second thin electrospun membrane of the first device unit, and wherein inlet and outlet conduits of the first device unit are in fluid connection with the respective inlet and outlet conduits of the second device unit.

In another aspect, the invention is directed to a process for manufacturing an intravascular artificial pancreas, comprising the steps of: (A) generating a first thin nanofibrous membrane by electrospinning a polymer containing solution; (B) depositing a sacrificial substrate in the form of at least one islet chamber upon a first side of the thin nanofibrous membrane; (C) depositing a plurality of sacrificial substrates in the form of at least a plurality of first vascular channels upon a second side of the thin nanofibrous membrane, wherein the sacrificial substrates in the form of first vascular channels each have a first end and a second end, wherein first ends are connected to a first input conduit and second ends are connected to a first outlet conduit; (D) encasing at least the thin nanofibrous membrane having the sacrificial substrates of step (B) and (C) deposited thereon within an encasement matrix; (E) removing the sacrificial substrates of steps (B) and (C) to provide a first vascular network and at least one islet chamber; and (F) filling the at least one islet chamber with an islet chamber matrix comprising islets and/or beta cells capable of producing proinsulin peptide and/or glucagon.

In some embodiments, the process further comprises: (C)(i) generating a second thin nanofibrous membrane by electrospinning a polymer containing solution over the sacrificial substrate in the form of at least one islet chamber; and (C)(ii) depositing a plurality of sacrificial substrates in the form of a plurality of second vascular channels upon a side of the second thin nanofibrous membrane opposite the side in contact with the sacrificial substrate in the form of at least one islet chamber, wherein the sacrificial substrates in the form of second vascular channels each have a first end and a second end, wherein the first ends are connected to a second inlet conduit and the second ends are connected to a second outlet conduit; wherein step (D) comprises encasing the second nanofibrous membrane, sacrificial substrates, the second inlet conduit and the second outlet conduit of (C)(i)-(ii) together with the first thin nanofibrous membrane, sacrificial substrates, first inlet conduit, and first outlet conduit of (A)-(C) within an encasement matrix; and wherein step (E) further comprises removing the sacrificial substrates of (C)(ii) to provide a second vascular network.

In some embodiments, the process further comprises (G) introducing a suspension of endothelial cells into the first vascular network and/or the second vascular network, and after a period time, flipping the device 180 degrees.

In some embodiments, the process further comprises the step (C)(iii) generating a third thin nanofibrous membrane by electrospinning a polymer containing solution over the plurality of sacrificial substrates formed in step (C); wherein step (D) further comprises encasing the third nanofibrous membrane, second nanofibrous membrane, sacrificial substrates, the second inlet conduit and the second outlet conduit of (C)(i)-(ii) together with the first thin nanofibrous membrane, sacrificial substrates, first inlet conduit, and first outlet conduit of (A)-(C) within an encasement matrix.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 depicts blood flow rates per tissue weight for native islets as well as common clinical transplantation sites.

FIGS. 2A-2E depict designs for pancreas scaffolds. FIG. 2A depicts a small-scale device that contains a single islet channel with a single vascular channel. FIG. 2B depicts a single-layer full-pattern device containing 50 vascular channels fed by a tapered distribution network. FIG. 2C depicts a cross-sectional view of a 2-double-layer full-scale design containing 200 total channels. FIG. 2D is an image of the 2-double-layer full-scale device. FIG. 2E depicts the vascular layer and islet layer of the 2-double-layer full-scale device.

FIGS. 3A-3C depict computational modeling and implant cell seeding of the pancreas scaffold. FIG. 3A depict computational models of shear stress and fluid pressure when cell culture media or blood is passed through the vascular and islet layers. FIG. 3B is an overview of the pancreas scaffold, with the section of the pancreas scaffold shown in FIG. 3C, indicated. FIG. 3C depicts a cross-sectional point of view of the vascular and islet layers after 2 weeks of in vitro perfusion, demonstrating good cell distribution and durability of the endothelial layer in the vascular channels.

FIGS. 4A-4C depict pancreas scaffold glucose responsiveness of rat islets. FIG. 4A demonstrates that rat islets loaded into the pancreas scaffold were glucose responsive on day 1, and positive for insulin and glucagon on day 4. FIG. 4B depicts stimulation index of pancreas scaffold, while FIG. 4C depicts stimulation index of a conventional suspension culture.

FIGS. 5A-5H depict various aspects of the development of iPS derived insulin producing cells in vitro and in scaffold.

FIGS. 6A-6D depict results of experiments conducted in pigs by implanting a one-layer intravascular artificial pancreas device by connecting it to a central venous catheter and perfusing it with pig blood.

FIGS. 7A-7H depict results from in vitro experiments with a two-double layer intravascular artificial pancreas device and luciferase secreting cells.

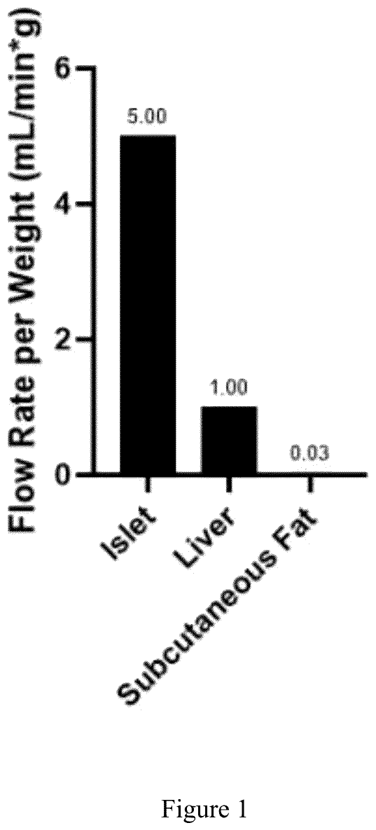

### DETAILED DESCRIPTION OF THE INVENTION

Pancreatic islets are one of the most highly perfused organs in the human body, however extraportal transplantation sites and encapsulation devices often prevent a high level of vascularization from occurring in transplanted islets. Described herein are intravascular artificial devices designed to support glucose sensing and insulin secretion across a mechanical and endothelial based immune barrier, to provide immediate blood supply at physiologic levels after implantation, and thereby support beta-cell engraftment and long-term function. The scaffold design is flexible, allowing devices to be made to a size that is useful for an experiment or in the future for a particular patient. The pattern has been designed to provide the shear rates required for a healthy endothelium, using different flow rates with media and blood to account for the difference in viscosity. Rat islets demonstrated glucose responsiveness in scaffold in vitro. Acute and first survival pig anastomosis experiments, showed proof-of-concept into the functional capability of an embodiments of the device. Finally, an experiment involving HEK-Lucia cells as a model cell that secretes a product with a similar molecular weight to insulin in response to a soluble signal demonstrated full scale scaffolds as described herein support a highly proliferative cell type in vitro.

An estimated full human dose of islets is often cited as 500,000 islet equivalents (IEQ, ˜1000-2000 cells, ˜150 μm diameter). Although perhaps not clinically necessary in the future, the inventors have designed the full-scale device to accommodate ˜660,000 IEQs. If spread out in a single layer, assuming square packing; this number of islet equivalents would require a large surface area of at least 145 cm². Thus, the inventors set out to design a compact device which houses a large dose of islets/beta-cells without sacrificing perfusion of the islets/beta-cells contained therein.

In one aspect, the invention is directed to a compact device capable of producing an amount of insulin sufficient to control glucose in a human subject. In some embodiments, the device comprises a plurality of first vascular channels each having a first end and a second end, wherein each of the first ends of the plurality of vascular channels connect to a first input conduit and each of the second ends of the plurality of vascular channels connect to a first output conduit, thereby forming a first vascular channel network; pancreatic islet and/or beta cells disposed within at least one islet chamber; and a thin membrane disposed as a biomolecule and gas permeable interface between the plurality of vascular channels and a first side of the at least one islet chamber, wherein the plurality of vascular channels and the first side of the at least one islet chamber are juxtaposed from each other across the thin membrane. This arrangement permits exchange of biomolecules and gases between the pancreatic islet and/or beta cells disposed within the at least one islet chamber and the plurality of vascular channels. In some embodiments, the device contains at least 2, 3, 4, 5, 6, 7, 8, 9, 10, 11, 12, 13, 14, 15, 16, 17, 18, 19, 20, 25, 30, 40, 50, 60, 70, 80, 90, 100, 110, 120, 130, 140, 150, 160, 170, 180, 190, 200, 225, 250, 275, 300, 350, 400, 450, 500, 750, 1000, 2000, or 10000 total channels. In some embodiments the device contains 10-1000, 15-500, 20-300, 25-200, 30-160, or 40-80 vascular channels. In some embodiments, the device contains between 2-200, 4-150, 10-80, 20-60, 60-180 or 30-80 islet channels.

The diameter and cross-sectional profile of the vascular channels and the at least one islet chamber are not particularly limited and may comprise diameters and cross-sectional profiles known to those of ordinary skill in this field or as specifically described herein. In some embodiments, the islet chamber and vascular channels have a cross-sectional profile that is round, oval, square, rectangular, or a profile that is concave, widened, or flattened on one or more sides. In some embodiments, the at least one islet chamber comprises a plurality of islet chambers, wherein the islet chambers are formed as islet channels. In some embodiments, the islet channels and/or the vascular channels have a widened cross-sectional profile, wherein the profile is elongated horizontally. In some embodiments, the islet channels and/or the vascular channels have a flattened cross-sectional profile, wherein the profile is elongated horizontally, but reduced vertically. When the islet and/or vascular channels are provided with a widened or flattened cross-sectional profile, it is beneficial to juxtapose the elongated sides of the islet and/or vascular channels across the membrane from its counterpart channel to provide greater surface area for exchange of biomolecules and gases across the membrane.

In some embodiments, the diameter of the islet channel is greater than the diameter of the vascular channels. In some embodiments, the diameter of the islet channel is equal in size to the diameter of the vascular channels. In other embodiments, the diameter of the islet channel is smaller than the diameter of the vascular channels. For purposes of measuring the inside diameter of a vascular or islet channel which is not circular, the diameter is the longest distance that can be measured between any two opposite points on the channel wall. In some embodiments, the diameter can individually include diameters of 100 mm, 50 mm, 10 mm, 5 mm, 1 mm, 500 μm, 50 μm, 10 μm, 5 μm, 3 μm, 1 μm, 0.5 μm, 0.1 μm, 0.05 μm, 0.02 μm, or 0.01 μm. In certain embodiments, the islet channel and/or the vascular channel are microchannels with a diameter of 900 μm, 800 μm, 750 μm, 700 μm, 650 μm, 625 μm, 600 μm, 575 μm, 550 μm, 525 μm, 500 μm, 475 μm, 450 μm, 425 μm, 400 μm, 375 μm, 350 μm, 325 μm, 300 μm, 275 μm, 250 μm, 225 μm, 200 μm, 175 μm, 150 μm, 125 μm, 100 μm, 75 μm, 50 μm, or 25 μm. In some embodiments, the smallest distance that can be measured between any two opposite points on the channel wall is about 10 μm, 15 μm, 17 μm, 20 μm, 22 μm, 25 μm, 27 μm, 30 μm, 33 μm, 35 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 400 μm, 500 μm, 600 μm, or 700 μm.

In some embodiments, at least two vascular channels interface with and are juxtaposed across from a first side of an islet chamber and/or channel through a thin membrane. In some embodiments, three vascular channels interface with and are juxtaposed across from an islet chamber and/or channel through one or more thin membranes. In some embodiments, four vascular channels interface with and are juxtaposed across from an islet chamber and/or channel through one or more thin membranes. In some embodiments, five, six, seven, eight, nine, ten, eleven, twelve, thirteen, fourteen, fifteen, sixteen, seventeen, eighteen, nineteen, twenty, thirty, forty, fifty or more vascular channels interface with and are juxtaposed across from an islet chamber and/or channel through one or more thin membranes. In some embodiments, each islet channel has a majority of the space surrounding it covered by one or more vascular channels, thereby participating in productive diffusion. In some embodiments, at least 55%, at least 60%, at least 65%, at least 70%, at least 75%, at least 80%, at least 85%, at least 90%, at least 95% or substantially all of the surface area of a vascular channel interfaces with vascular channels through one or more thin membranes.

In some embodiments, it is beneficial to provide a flattened vascular channel to provide increased surface area for interfacing with one or more vascular channels while conserving space in the device. Thus, in some embodiments, the vascular channel is flattened and multiple vascular channels interface with an elongated top side of the vascular channel through a thin membrane. In some embodiments, the vascular channel is flattened and multiple vascular channels interface with an elongated bottom side of the vascular channel through a thin membrane. In some embodiments multiple vascular channels interface with an elongated top side of the vascular channel and multiple vascular channels interface with the elongated bottom side of the vascular channel through one or more thin membranes.

In some embodiments, it is beneficial to provide a vascular channel that is flattened and can only accommodate a small number of islets/beta-cell clusters stacked vertically therein. This ensures that each islet/beta-cell is close to the membrane, facilitating quick diffusion or exchange of glucose, insulin, oxygen and other biomolecules across the membrane to each adjacent islet/beta-cell. In some embodiments, the vascular channel is flattened, and the height of the vascular channel is only capable of fitting two, three, four, five or six islets/beta-cell clusters stacked vertically therein. In some embodiments, the height of the vascular channel is only capable of accommodating two islets/beta-cell cluster stacked vertically therein. In some embodiments, the height of the vascular channel is approximately 20-300 μm, 25-250 μm, 30-225 μm, 25-150 am, or 75-300 μm.

In other aspects, the invention is directed to a device which comprises one or more thin membranes or films as a basement membrane. In some embodiments, the one or more thin membranes or films form the wall of each one of a plurality of vascular channels, islet chambers or islet channels. In some embodiments, a plurality of vascular channels are juxtaposed across the thin membrane or film from an islet chamber or islet channel. In some embodiments the thin membrane or film serves as a biomolecule and/or gas permeable interface between a plurality of vascular channels and at least one islet chamber or islet channel.

The composition of the thin membrane or film is not particularly limited and may be any composition suitable for forming a thin membrane or film with the desired porosity and mechanical strength necessary to withstand shear stress and pressure exerted during the manufacturing process and use of the device in vivo. This includes the flow of a biological fluid therethrough under normal hemodynamic pressure, such as a pressure of at least 60 mmHg. In some embodiments, the plurality of membranes are each generated by chemical or physical thin film deposition, atomization, spraying, electrospinning, dip coating, or gelation of a solution comprising decellularized tissue, gelatin, gelatin composites, collagen, fibrin, hydrogel, hydrogel composites, chitosan, nitrocellulose, polylactic acid, polycaprolactone, extra-cellular matrix that has been liquefied or homogenized, or mixtures thereof, in a thin film layer followed by curing, crosslinking, polymerizing, drying, or gelating the solution to form a membrane layer. In some embodiments, the thin membrane or film has a thickness of 0.5-30, 1-20, 4-15, 7-25, 8-13, 9-20, or 10-14 micrometers. In some embodiments, the thin membrane or film has a thickness of approximately 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 micrometers.

In some embodiments, the membrane solution further comprises a porogen homogenously mixed therein. In some embodiments, the porogen is a self-assembling tri-block copolymer. In some embodiments, the self-assembling tri-block copolymer is a poloxamer formulation, preferably Pluronic F127 at a concentration of 1-40% wt.

In some embodiments, the membrane solution further comprises one or more agents modifying the mechanical or biological properties of the one or more membranes. In some embodiments, the one or more agents are selected from glycerin, sorbitol, propylene glycol, plasticizers, fibers or other longitudinal elements, and encapsulated growth factors. In some embodiments, the membrane solution further comprises fibers, nanotubes, or other longitudinally oriented materials in order to provide improved mechanical properties. These fibers can be mixed into the membrane solution prior to fabrication in order evenly distribute the fibers throughout the membrane. Alternatively, these fibers can be deposited or integrated onto the membrane after fabrication through techniques such as electrospinning, 3D printing, or other techniques. The fibers may be homogenously distributed throughout the membrane or may be distributed in an organized manner to provide heterogenous mechanical properties for the membrane. In some embodiments, the method of generating a thin film layer is repeated one or more times to generate a membrane or membranes having two or more membrane layers. In some embodiments, the two or more layers are generated from solutions having different components, agents and/or concentrations. In some embodiments, at least one of the plurality of membranes are treated to remove the porogen, thereby forming pores in the membrane.

In some embodiments, the membrane solution comprises 3-35 wt % of gelatin or a gelatin-polymer composite. In some embodiments, the thin film layer is crosslinked with a solution comprising glutaraldehyde, transglutaminase, or other crosslinking enzymes or molecules. known to an ordinarily skilled technician in the field or those which are described herein.

In some embodiments, the thin membrane is a fibrous membrane comprising electrospun fibers. In some embodiments, the fibrous membrane material comprises electrospun fibers comprising a binary, ternary, quaternary or quinary mixture of materials. In some embodiments, the fibrous membrane material comprises electrospun fibers comprising a first component selected from the group consisting of polycaprolactone, polyethylene glycol, and polyethylene glycol diacrylate, and a second component selected from the group consisting of gelatin, collagen and fibrin. The ratio of the first component to the second component include ratios of 1:1, 1:2, 1:3, 1:4, 1:5, 1:6, 1:7, 1:8, 1:9, 10:95, 1:10, 2:1, 3:1, 4:1, 5:1, 6:1, 7:1, 8:1, 9:1, 95:10, or 10:1. In some embodiments, the first and second component are polycaprolactone/gelatin, polycaprolactone/collagen, polycaprolactone/fibrin, polyethylene glycol diacrylate/gelatin, polyethylene glycol diacrylate/collagen, polyethylene glycol diacrylate/fibrin, polyethylene glycol/gelatin, polyethylene glycol/collagen, or polyethylene glycol/fibrin. Particularly preferred mixtures include a binary mixture of collagen and polycaprolactone. In one particularly preferred embodiment the collagen takes the form of bovine, porcine or fish gelatin with a molecular weight of 15-400 kDa. In some embodiments, the gelatin will have a bloom value of 30-300, a bloom value of 40-100, a bloom value of 100-200 or a bloom value of 200-280. Additionally, the gelatin is cross-linked. In another preferred embodiment, the polycaprolactone utilized to form electrospun fibers in the thin membrane has a molecular weight of 10-100 kDa, 25-80 kDa or 30-60 kDa. In some embodiments, the membrane is formed from the mixture of polycaprolactone and gelatin in a 1:1 ratio. In some embodiments, the fibrous electrospun membrane has a thickness of 0.5-30, 1-20, 4-15, 7-25, 8-13, 9-20, or 10-14 micrometers. In some embodiments, the fibrous electrospun membrane has a thickness of approximately 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 micrometers.

In some embodiments, the thin fibrous electrospun membrane material has been subjected to one or more post-fabrication treatments selected from compression, annealing, chemical crosslinking, stretching, drawing, heat treatment, and solvent welding, whereby the treated fibrous electrospun membrane material is imparted enhanced mechanical properties compared to a fibrous electrospun membrane material not receiving one or more of the post-fabrication treatments. In some embodiments, the enhanced mechanical properties are selected from the group consisting of enhanced tensile strength, enhanced tensile modulus, enhanced abrasion resistance, enhanced thermal stability, enhanced elongation at break, enhanced hardness, enhanced crystallinity and combinations thereof. In some embodiments, the post-fabrication treatment comprises solvent welding. In some embodiments, the solvent welding is conducted in the presence of pressure imparted by opposing support substrates. In some embodiments, post-fabrication treatment comprises heat treatment in combination with pressure imparted by opposing support substrates.

In some embodiments, the electrospinning is carried out in close-proximity to the substrate/collector plate to be coated with electrospun fibers. In some embodiments the distance between tip and collector plate is less than 15 cm, less than 12 cm, less than 10 cm, less than 9 cm, less than 8 cm, less than 7 cm, less than 6 cm, less than 5 cm, less than 4 cm, less than 3 cm, or less than 2 cm. In some embodiments, the diameter of the inner aperture of the tip, the feed rate of the fiber precursor solution or melt, and/or the concentration of the polymer in the precursor solute or melt is reduced to produce a smaller diameter fiber. Additionally, the applied voltage may be adjusted to reduce the diameter of the formed fiber, such as by causing increased stretching or drawing of the ejected precursor solution or melt before deposition on the collector substrate. In some embodiments, volatility of any solvent for use in dissolving the fiber precursor material is carefully considered, along with other spinning parameters, to ensure proper fiber formation and deposition is achieved in the finished fibrous membrane. In certain embodiments, reducing the diameter of the fiber permits the evaporation of the solvent in the precursor solution, or cooling and hardening of the precursor melt, before arriving on the sacrificial substrate/collector plate as solid fiber. This can be especially important when distance between tip and sacrificial substrate/collector plate is reduced or small, as required in certain embodiments of the invention. In alternative embodiments, the feed rate, aperture size of the needle and applied voltage is selected to provide semi-solid fibers which anneal to each other upon deposition on the substrate/collector plate, thus reducing the duration of a post-fabrication treatment step, or even permitting the elimination of post-fabrication treatment altogether.

In some embodiments, the fiber diameter is less than 10 μm, less than 9 μm, less than 8 μm, less than 7 μm, less than 6 μm, less than 5 μm, less than 4 μm, less than 3 μm, less than 2 μm, or less than 1 μm. In some embodiments, a majority of fibers present in the fibrous membrane are nanofibers, with a diameter of 950 nm or less, 800 nm or less, 600 nm or less, 450 nm or less, or 200 nm or less. In some embodiments, the majority of fibers in the fibrous membrane are nanofibers having a diameter between approximately 100 nm and 750 nm, between approximately 100 nm and 500 nm, or between approximately 250 nm and 800 nm. In some embodiments, the fibrous membrane comprises fibers with a diameter of greater than 950 nm, greater than 2 μm, greater than 3 μm, greater than 4 μm, greater than 5 μm or more. In some embodiments, the diameter of the electrospun fiber is at least 5 μm, at least 6 μm, at least 7 μm, at least 8 μm, at least 9 μm or more.

In some embodiments, the thin membrane or film comprises pores of sufficient size to enable diffusion of one or more biologically relevant molecules. The pore size of the pores in the membrane or film may be any suitable size and is not limited. In one embodiment, the average or median pore size diameter is about 0.05 to about 0.6 μm. In another embodiment, the average or median pore size diameter is about 0.05 μm, 0.1 μm, 0.15 μm, 0.2 μm, 0.25 μm, 0.3 μm, 0.35 μm, 0.4 μm, 0.45 μm, 0.5 μm, 0.55 μm or about 0.6 μm. The porosity of the nanofiber membrane (Pnm), obtained by dividing volume of voids (Vv) by the total volume of membrane or film measured (VTvm) (P=Vv/VTvm*100%), may be any suitable porosity and is not limited. In some embodiments the porosity is at least 5%, 10%, 20%, 30%, 40%, 50%, 60%, 70%, 80%, 90% or more. In other embodiments the porosity is between 20-80%, 30-70% or 40-60%.

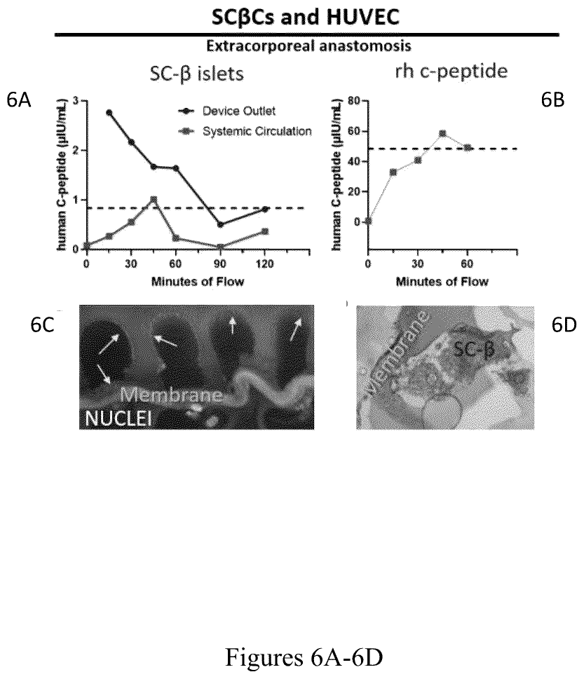

In another aspect, the invention is directed to the provision of a compact device, without sacrificing perfusion of a large dose of islets. Thus, in some embodiments, the device comprises multiple functional units that are stacked together. In some embodiments, a functional unit of the device comprises a vascular layer comprising a plurality of vascular channels, an islet layer comprising at least one islet chamber or a plurality of islet channels, and a thin membrane or film which serves as a biomolecule and gas permeable interface between the plurality of vascular channels of the vascular layer and the islet chamber or the plurality of islet channels of the islet layer. In some embodiments, a functional unit comprises a first and second vascular layer, each comprising a plurality of vascular channels which are disposed on either side of an islet layer comprising at least one islet chamber, or a plurality of islet channels, wherein a thin membrane or film is placed between the first and second vascular layers and the opposing sides of the islet layer, to provide a double vascular layer functional unit. In some embodiments, two, three, four, five, six, seven, eight, nine, ten, eleven, twelve or more functional units are combined to provide a device capable of perfusing a large dose of pancreatic islets/beta-cells. Extrapolation from the ˜15k Hypol-SCβCs IEQs loaded (acute single layer anastomosis, FIG. 6) to a 500,000 IEQ human dose, would have produced approximately 100 units of insulin, more than enough to control glucose in patients. Thus, in some embodiments, the device is a 2 unit-double vascular layer device capable of housing 500,000 IEQs. Therefore, the device has the potential to function for pre-IND experiments in the current form, if it is housing the cell number that a 2 unit, double vascular layer device can accommodate. This device design is enabled by layering to achieve the requisite functional membrane area in a reasonable footprint.

In some embodiments, the current device has a footprint similar to the size of a smartphone (˜10 cm wide, ˜6 cm long, ˜1 cm thick). In some embodiments, the width of the device measures approximately 4-20 cm, 8-15 cm, 6-13 cm, or 8-12 cm. In some embodiments, the length of the device measures approximately 3-18 cm, 4-15 cm, 5-8 cm, or 6-12 cm. In some embodiments, the device thickness measures approximately 0.25-3 cm, 2-6 cm, 0.5-2 cm, 0.75-3 cm, or 0.25-1 cm. At a high level, layering techniques connect the layer inputs to a common input and layer outputs to a common output.

In certain aspects, the device is designed to enable functional maturation before implantation, which is a key feature when stem cell derived insulin producing cells are used. The final function can be measured and tailored before the device is implanted.

Clotting is a major concern for any blood contacting material, and is a concern for the IVAP. The primary safeguard against clot formation is endothelializing the IVAP vascular channels. No acute clotting reactions were found in the short-term (<4 hours) pig anastomosis experiments.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

As used herein “A and/or B”, where A and B are different claim terms, generally means at least one of A, B, or both A and B. For example, one sequence which is complementary to and/or hybridizes to another sequence includes (i) one sequence which is complementary to the other sequence even though the one sequence may not necessarily hybridize to the other sequence under all conditions, (ii) one sequence which hybridizes to the other sequence even if the one sequence is not perfectly complementary to the other sequence, and (iii) sequences which are both complementary to and hybridize to the other sequence.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

### Materials and Methods

### Design of Scaffolds

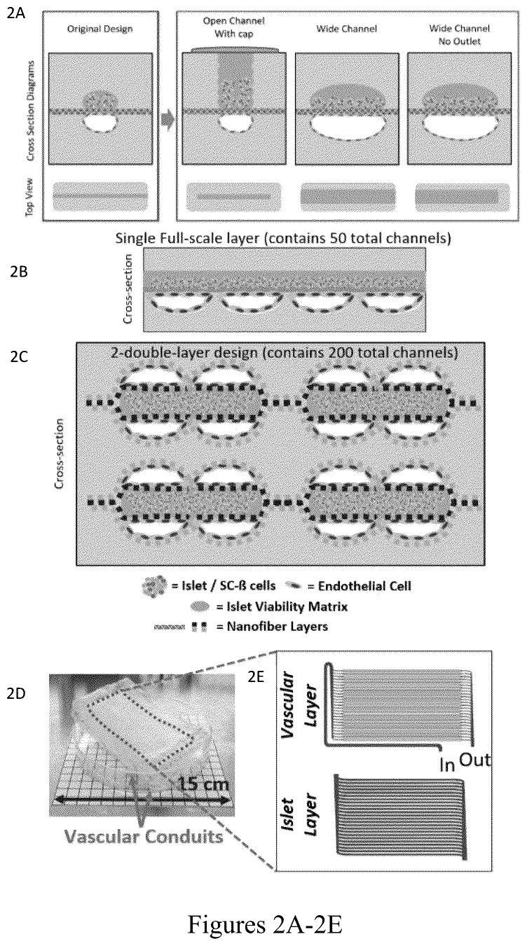

Pancreas scaffolds were made in 3 categories: small single-channel devices, single-layer full-pattern devices, and multi-layer full-scale devices. The small-scale devices evolved through several iterations, that all contain a single islet channel with a single vascular channel (FIG. 2A), facilitating preliminary testing. A single-layer full-pattern device contains 50 vascular channels fed by a tapered distribution network, with the islet compartment having separate access (FIG. 2B). Finally, to reach >500k IEQ capacity and learning from experience with loading multi-layer implants, multi-layer full-scale devices contain 2 double layers, where an islet layer that can accommodate 2 islets stacked vertically in wide channels with a vascular layer above and below to service the adjacent cells (FIGS. 2C, 2D). The vascular and islet sections were accessed by conduits that connect to all layers of that type. There were 2 islet conduits on opposite corners. For the vascular conduits, a wrap around channel was designed to bring both arterial and venous conduits to the same side for surgical anastomosis. To facilitate cell seeding, a third vascular conduit at the opposite corner was used, which was closed after seeding. This design can hold about 660k IEQs.

### Electrospinning of Membranes

Polycaprolactone (80 kDa, Sigma 440744; or Polysciences 26290) and gelatin (type A 300 bloom, Sigma G2500) were both dissolved at 9% w/v each (for a total of 18% polymer) in an acetic acid (AA) formic acid (FA) mixture (8:1 AA:FA). Membranes were electrospun in a custom bipolar electrospinning setup that had environmental control, automated stage movement, custom voltage control, dual syringe pumps all in a vented enclosure. The following parameters were used: 12 kV, 15 mA, opposite polarity for opposing nozzles, ˜0.5-1.5 uL/min, 30-40% RH and 24-26 C, ˜10-15 min duration adjusted to achieve ˜10-14 um membrane thickness. Membranes were either directly used or stored dry until use.

### Construction of Scaffolds

The overall workflow, with adjustments for the particular scaffold type, was as follows. A PCL-gelatin blend membrane was electrospun on a PLA 3-D printed frame as detailed above. The vascular and islet patterns were printed on the membrane with Pluronic F127 hydrogel (28%), conduits were placed and connected to the pattern with F127 by hand. Fabricated pattern layers were sterilized with ETO. Inside a biological safety cabinet (BSC), molds that contain the membranes were filled with a warm T-gase gelatin mixture (1:10 ratio of gelatin (12.25%) and transglutaminase (25%)) and scaffold assembly proceeded by alternating gelatin and prepared membranes as needed. After initial thermosetting as the cast gelatin cools, the scaffold with frames were submerged in phosphate buffered saline (PBS) supplemented with antibiotics and sealed in a sterile container which was stored in the 4 C. After 24-48 hours, the PLA molds and frames were removed in a BSC using sterile technique and the scaffold, still in the PBS with antibiotics was placed in a 37 C incubator to allow gelatin contraction to occur. After 24-48 hours the scaffold was returned to 4 C until loading of cells.

### HEK Cells

Human embryonic kidney (HEK) cells that secrete a soluble luciferin (Lucia) in response to IFN in the culture media (hkl-null, Invivogen, San Diego, CA, USA) were used as a surrogate for insulin producing cells for scaffold testing. After being grown in monolayer as described by Invivogen, cells were aggregated in non-adherent cell culture dishes with orbital rotation at ˜50-100 rotations per minute, allowing ˜7 days for aggregate formation before loading into scaffolds. HEK aggregates were then treated like pancreatic islets for loading into scaffolds.

### Stem-Cell Derived Insulin Producing Cells

Insulin producing cells have been differentiated following the Millman Lab adherent protocol [29,30] and suggested modifications to improve the differentiation for a particular cell line noted in the published work, with one exception. The pH of the enriched serum-free medium (ESFM) has been adjusted to be closer to pH 7, with an effect on the beta cell insulin content and glucose responsiveness that is at least as good as other modifications assayed. The starting induced pluripotent stem-cells (iPS) are double-knock-out HLA Class I&II null (B2M−/−, CIITA−/−) (Hypo1), have been provided by our collaborator Pancella (Toronto, Ontario).

### Isolation of Rat Islets

The isolation procedure was similar to published methods. Briefly, rats were individually euthanized with carbon dioxide asphyxiation, and laid supine. A wide incision in a V shape was made from rib-cage to lower abdomen to rib-cage to expose the peritoneal cavity. The common bile duct was obstructed where it enters the intestine, and a collagenase solution was injected into the common bile duct in the region near the liver. After the pancreas was inflated with enzyme it was carefully removed and placed on ice in a centrifuge tube. Once all animals were finished, digestion proceeded in a 37 C waterbath and the centrifuge tubes were shaken vigorously for 10 seconds. Tubes were immediately placed on ice and the digested tissue was repeatedly washed, purified with a Histopaque 2-layer gradient and washed before plating.

### Loading Islets or Cell Clusters

A prepared scaffold was flushed using all conduits with sterile PBS repeatedly to remove remaining traces of F127. The scaffold was then drained of PBS. The cell clusters in the islet chambers were embedded in a collagen based matrix (Islet Viability Matrix(IVM) [31]). Cold “IVM Base” was mixed with cold collagen type 1, to create IVM. The cell clusters were carefully resuspended in IVM. The cell cluster suspension was loaded immediately into the islet chambers of the scaffold, the vascular channels were flushed with PBS and the scaffold was placed in the 37 C incubator in a sealed container or bioreactor for 2 hours to allow the IVM to polymerize. (Note: in the single layer designs it is desirable to have islets at the membrane so the scaffold was kept cold for 1 hour to allow islets to settle to the membrane by gravity before polymerization at 37 C.)

### Seeding Endothelial Cells and Scaffold Culture

After the IVM/cluster suspension had polymerized in a scaffold, it was mounted in a bioreactor by attaching the vascular conduits to the ports on the bioreactor, allowing a roller pump driven flow loop to perfuse the scaffold during culture. Stopcocks were mounted on the outside of the bioreactor to allow interaction with the fluid path. An endothelial cell suspension was pushed into the vascular channels through the stopcock, the closed bioreactor was then turned over and placed in the 37 C incubator for 1 hour, designated as Seeding 1. Seeding 2 proceed the same way, only the bioreactor was left “right-side-up” during the 1 hour incubation. In this way, cells were seeded to both sides of the channels. After both seedings are complete, flow with media is initiated (single channel scaffold: 0.15 mL/min, full single layer: 5 mL/min, 2-double layer: 20 mL/min).

We have endothelialized the 2-double-layer scaffolds with glomerular microvascular endothelial cells (GMEC, for in vitro experiments) and (HUVEC, for in vivo experiments). More than a confluent monolayer based on the calculated surface area (we used ˜2×10\hat{ }6 cells per full length channel) were seeded to decrease the time to confluence.

### Computational Fluid Dynamics

To increase the speed with which we were able to validate and iterate on blood vessel patterns a validated computational fluid dynamics (CFD) model was required. For media we utilized a Newtonian fluid model (assumed to be equivalent to water for model). For blood, a pressure driven flow with a non-Newtonian carreau fluid model[32] was used. The model was run in Ansys Fluent 2022 R1, with a laminar flow assumption.

### GSIS Scaffold Protocol

After seeding the scaffold and perfusion culturing in maturation media overnight, the device will be perfused with Krebs-Ringer's Buffer in the following sequence. 1) Low glucose (4 hr), 2) Low glucose (2 hr), 3) High glucose (2 hr), 4) Low glucose (4 hr). Samples will be collected on an hourly basis throughout the 12-hour experiment and frozen for assay using a human insulin or c-peptide enzyme linked immuno-assay (ELISA, Mercodia). Stimulation index (SI) is calculated by dividing the insulin secreted in high glucose by the insulin secreted in low glucose.

### Large Animal Studies

Porcine recipients were anesthetized and dosed heparin to maintain a long clotting time (ACT), at CBSET in Lexington, MA. For this terminal experiment, central lines were placed and blood flow was controlled through the implant in a bioreactor by external pump. Blood samples were collected in EDTA coated tubes and maintained on ice until centrifugation to isolate serum could be done. Serum samples were then frozen at −20 C until quantification by ELISA (as described for GSIS above).

### Histology and Analysis

Scaffolds were fixed by submersion in buffered formalin (Electron Microscopy Sciences), washed 3X with PBS and stored at 4 C. Sections of interest were soaked in sucrose, embedded in gelatin/sucrose and mounted on chucks for frozen sections. For large scaffolds (single or 2-double layer), six sections are made at 3 locations along the length of the channels (note it is 6 not 3 as the scaffold width is 2× that of standard histology cassettes). Samples were stained with DAPI or HOESCHT to identify nuclei. Immunostaining for c-peptide/insulin (NBP1-05433, Novus Biologicals, Centennial, CO, USA; 1:10 dilution) and glucagon (259A-18, Sigma-Aldrich, used as supplied) was completed on the rat islet scaffold sections. TUNEL staining was completed following manufacturer's directions (Roche 11684795910).

### Results

### Computational Modeling and Implant Cell Seeding

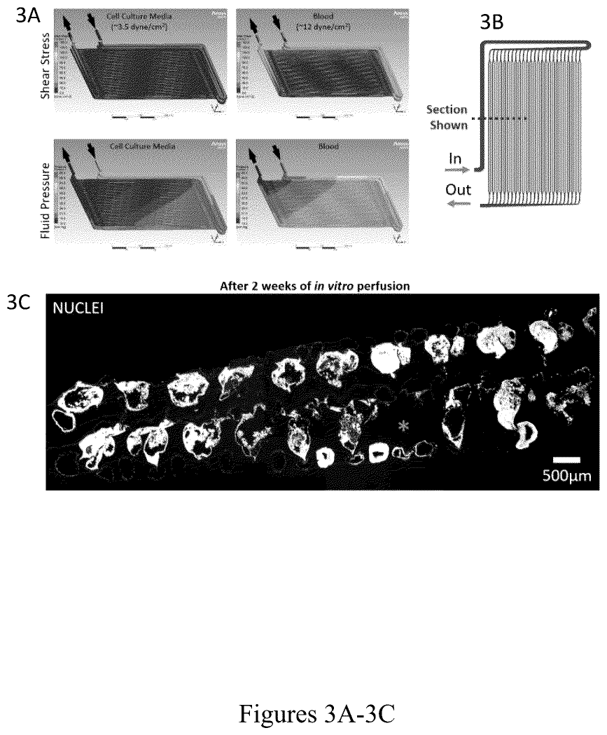

Having verified computationally predicted flow rates occur on the benchtop for water, we used in silico modeling to calculate flow rates required to reach shear rates within the range to maintain endothelial integrity. Modeling predicts an even distribution of flow (FIG. 3A). Our findings suggest good cell distribution when the pattern fidelity is high. Note the presence of only a single islet channel that was not seeded (asterisks) and the majority of vascular channels still having an endothelial layer after 2 weeks of in vitro perfusion (FIGS. 3B, 3C).

### Demonstration of Scaffold Glucose Responsiveness

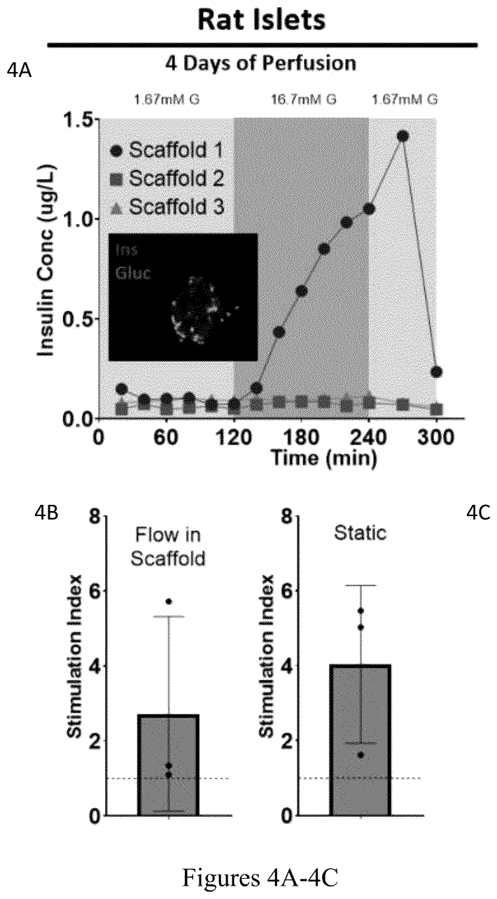

Using the best small-device design, rat islets loaded into scaffolds were positive for insulin and glucagon at 4 days of in vitro culture, after showing glucose responsiveness on day 1 (FIG. 4A). The best scaffold reached a stimulation index similar to the conventional culture islets, suggesting variability in the cell loading or islets, rather than a material incompatibility prevented all 3 from being clearly glucose responsive (FIGS. 4B, 4C).

### Development of iPS Derived Insulin Producing Cells

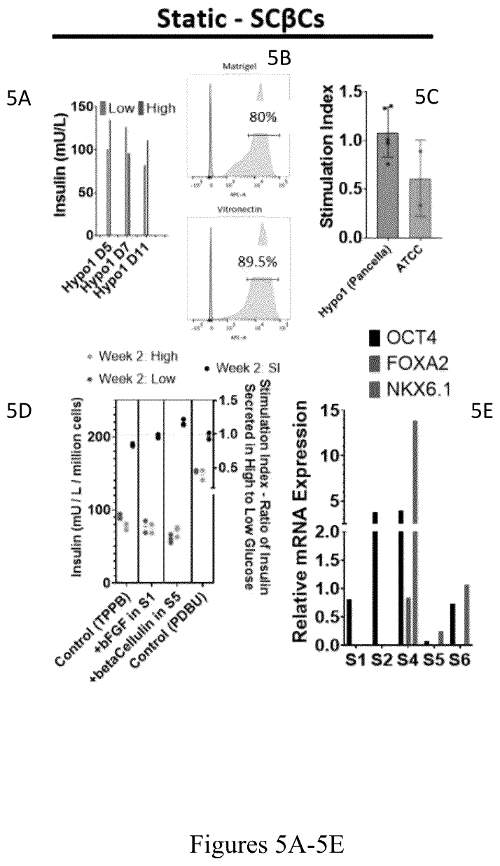

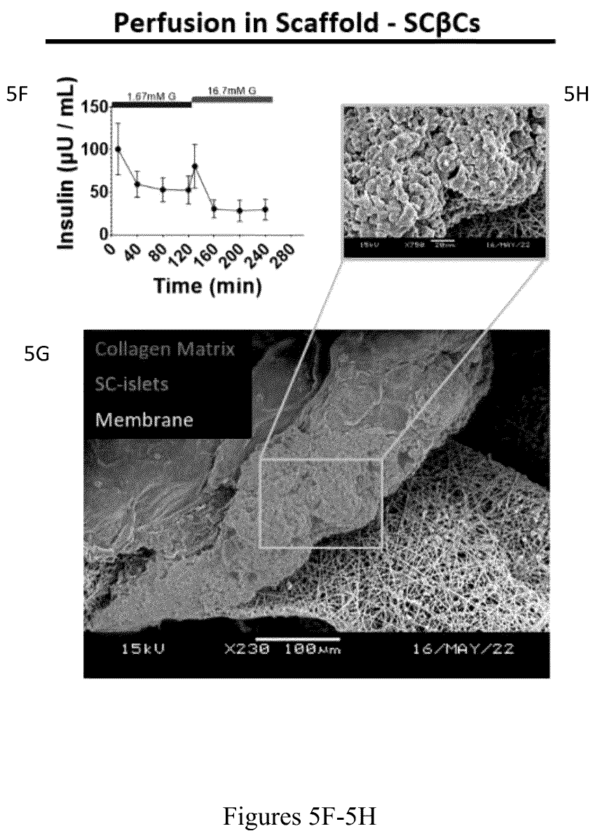

We have been tailoring the Millman Lab adherent protocol [29,30] to hypoimmune iPS cells (Hypol) using suggested additions to the chemical cocktails applied during certain stages of differentiation. Thus far the cells have been inconsistently glucose responsive (FIG. 5A), 80% PDX1+ on Matrigel and 90%+ on vitronectin (however vitronectin did not support cell attachment in the long-term) (FIG. 5B), but are better than a control stem-cell line from ATCC (FIG. 5C). Using PDBU instead of TPPB, addition of bFGF in S1 and addition of betacellulin in S5 of the protocol caused modest functional improvement for the Hypol SCβCs at 2 weeks of maturation (FIG. 5D). Preliminary mRNA expression from Hypol cells show continued presence of OCT4 cells throughout the differentiation stages; late and short expression of FOXA2; and temporally reasonable expression of NKX6.1 (FIG. 5E).

### iPS Beta-Cells in Scaffolds In Vitro and Extracorporeal

Cells from one batch were loaded into a small scaffold, did not appear glucose responsive (FIG. 5F), but maintained aggregate morphology in scaffold over a long period of time (FIGS. 5G, 5H). In a series of non-survival large animal experiments in porcine hosts two one-layer implants were connected to central venous catheters. When the implant contained Hypol SCβCs, and the vascular pattern was endothelialized with HUVEC and perfused by pig blood, we detected human c-peptide at the output of the device for the first hour and were able to detect human c-peptide at 45 mins in the porcine circulation (FIG. 6A). In a second implant, recombinant C-peptide also peaked at 45 minutes (FIG. 6B), demonstrating the device could deliver a larger dose that reached human fasting concentrations of 0.9 ng/mL c-peptide. The rate of insulin delivery with the Hypol SCβCs was 1.41 U/hr while it was 781 U/hr when the IVAP was loaded with recombinant c-peptide. A cellular lining in the vascular channels (FIG. 6C) and Hypol SCβCs were still evident in the islet channels (FIG. 6D) following blood flow.

### Two-Double Layer IVAP Cell Survival and Function In Vitro

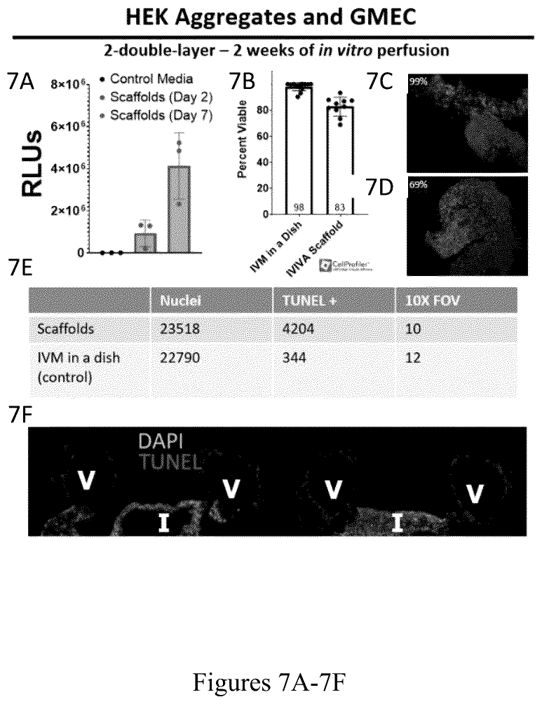

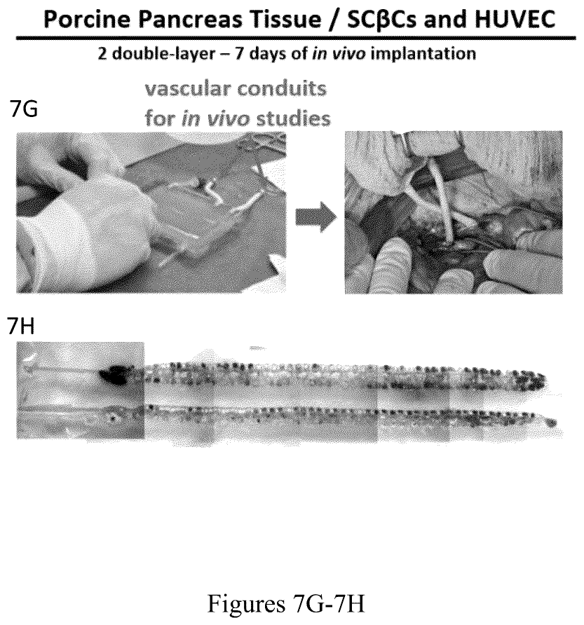

A series of experiments with luciferase secreting cells (Luciferase ˜24 kDa vs insulin ˜5.8 kDa) demonstrated diffusion into the outflow after IFN stimulation in the inflow in vitro at 2 and 7 days of perfusion culture in a 2-double-layer scaffold. Three 2-double-layer scaffolds were loaded with at least 1 mL packed cell pellet of HEK aggregates. All 3 scaffolds produced luciferase at 2 days and then an increased amount upon a second stimulation at 7 days (FIG. 7A). Luciferase continued to be produced by the scaffold (data not shown). Furthermore, following 2 weeks of perfusion culture viability was ˜80%, with a high degree of cellularity (FIGS. 7B-7F).

### Two-Double Layer IVAP In Vivo Anastomosis and Implantation

Two HUVEC endothelialized 2-double-layer IVAPs with vascular conduits were anastomosed to the aorta and vena cava (FIG. 7G). Blood was freely flowing in the vascular pattern upon physical examination of the grafts at the endpoint. The scaffold architecture fidelity was high post explant (FIG. 7H, blood presence in channels on the cut piece is not a complete representation of perfusion, as blood drains from channels when a chunk is removed from a scaffold). Both animals survived to the pre-determined endpoints of 1 and 7 days, showing proof of concept for the surgical model and the implant construction.

In summary, the devices disclosed herein provide permanent insulin independence and freedom from the risk of diabetic complications by allowing for full maturation before implantation and for immediate perfusion after implantation. The use of hypo-immune iPSC derived endothelial cells provide for biologic immune protection and prevent rejection by the recipient. As a better treatment for diabetes, the IVAP will decrease the burden on the healthcare system, and the number of people who exit the organ transplant waiting list by death.

### REFERENCES

[1] Vertex Pharmaceuticals Incorporated, Vertex Announces Positive Day 90 Data for the First Patient in the Phase 1/2 Clinical Trial Dosed With VX-880, a Novel Investigational Stem Cell-Derived Therapy for the Treatment of Type 1 Diabetes, Https://Investors.Vrtx.Com/News-Releases/News-Release-Details/Vertex-Announces-Positive-Day-90-Data-First-Patient-Phase-12. (2021).
[2] Health Resources and Services Administration, Organ Donation Statistics, Https://Www.Organdonor.Gov/Learn/Organ-Donation-Statistics. (n.d.).
[3] A. M. J. Shapiro, C. Ricordi, B. J. Hering, H. Auchincloss, R. Lindblad, R. P. Robertson, A. Secchi, M. D. Brendel, T. Berney, D. C. Brennan, E. Cagliero, R. Alejandro, E. A. Ryan, B. DiMercurio, P. Morel, K. S. Polonsky, J.-A. Reems, R. G. Bretzel, F. Bertuzzi, T. Froud, R. Kandaswamy, D. E. R. Sutherland, G. Eisenbarth, M. Segal, J. Preiksaitis, G. S. Korbutt, F. B. Barton, L. Viviano, V. Seyfert-Margolis, J. Bluestone, J. R. T. Lakey, International trial of the Edmonton protocol for islet transplantation, N Engl J Med. 355 (2006) 1318-1330. https://doi.org/10.1056/NEJMOA061267.
[4] E. D. Foster, N. D. Bridges, I. D. Feurer, T. L. Eggerman, L. G. Hunsicker, R. Alejandro, B. Begley, J. Cano, S. Carpentier, J. Hutchinson, C. P. Larsen, J. Moreno, M. Sears, N. A. Turgeon, D. Webster, J. F. Markmann, P. Al-saden, X. Chen, A. Hecyk, X. Luo, M. Molitch, N. Monson, E. Stuart, A. Wallia, L. Wang, S. Wang, X. Zhang, C. W. Czarniecki, J. S. Goldstein, A. Priore, M. A. Robien, E. Schneider, G. Arreaza-Rubin, N. Green, D. L. Bigam, P. Campbell, P. Dinyari, T. Kin, N. M. Kneteman, J. Lyon, A. Malcolm, D. O'Gorman, C. Onderka, R. Owen, R. Pawlick, B. Richer, S. Rosichuk, D. Sarman, A. Schroeder, P. A. Senior, A. M. J. Shapiro, L. Toth, V. Toth, W. Zhai, K. Johnson, J. McElroy, A. M. Posselt, M. Ramos, T. Rojas, P. G. Stock, G. Szot, B. Barbaro, J. Martellotto, J. Oberholzer, M. Qi, Y. Wang, L. Bayman, K. Chaloner, W. R. Clarke, J. S. Dillon, C. Diltz, G. C. Doelle, D. Ecklund, D. Feddersen, C. Jasperson, D.-E. Lafontant, T. Neill-Hudson, D. Nollen, J. Qidwai, H. Riss, T. Schwieger, J. Willits, J. Yankey, A. C. Corrales, R. Faradji, T. Froud, A. Alvarez Gil, E. Herrada, L. Inverardi, N. Kenyon, A. Khan, E. Linetsky, E. Peixoto, C. Ricordi, M. H. Abdulla, A. N. Balamurugan, M. D. Bellin, M. Brandenburg, J. v. Harmon, B. J. Hering, R. Kandaswamy, G. Loganathan, K. Mueller, K. K. Papas, J. Pedersen, J. J. Wilhelm, J. Witson, C. Dalton-Bakes, H. Fu, M. Kamoun, J. Kearns, Y. Li, C. Liu, E. Luning-Prak, Y. Luo, E. Markmann, Z. Min, A. Naji, M. Palanjian, M. R. Rickels, R. Shlansky-Goldberg, K. Vivek, A. S. Ziaie, D. B. Kaufman, O. Korsgren, Improved Health-Related Quality of Life in a Phase 3 Islet Transplantation Trial in Type 1 Diabetes Complicated by Severe Hypoglycemia, Diabetes Care. 41 (2018) 1001-1008. https://doi.org/10.2337/DC17-1779.
[5] D. Nyqvist, S. Speier, R. Rodriguez-Diaz, R. D. Molano, S. Lipovsek, M. Rupnik, A. Dicker, E. Ilegems, E. Zahr-Akrawi, J. Molina, M. Lopez-Cabeza, S. Villate, M. H. Abdulreda, C. Ricordi, A. Caicedo, A. Pileggi, P. O. Berggren, Donor Islet Endothelial Cells in Pancreatic Islet Revascularization, Diabetes. 60 (2011) 2571-2577. https://doi.org/10.2337/DB10-1711.
[6] T. M. Suszynski, J. J. Wilhelm, D. M. Radosevich, A. N. Balamurugan, D. E. R. Sutherland, G. J. Beilman, T. B. Dunn, S. Chinnakotla, T. L. Pruett, S. M. Vickers, B. J. Hering, K. K. Papas, M. D. Bellin, Islet Size Index as a Predictor of Outcomes in Clinical Islet Autotransplantation, Transplantation. 97 (2014) 1286. https://doi.org/10.1097/01.TP.0000441873.35383.1E.
[7] T. Desai, L. D. Shea, Advances in islet encapsulation technologies, Nature Reviews Drug Discovery 2016 16:5. 16 (2016) 338-350. https://doi.org/10.1038/nrd.2016.232.
[8] R. E. Gurlin, J. A. Giraldo, E. Latres, 3D Bioprinting and Translation of Beta Cell Replacement Therapies for Type 1 Diabetes, Tissue Eng Part B Rev. 27 (2021) 238-252. https://doi.org/10.1089/TEN.TEB.2020.0192.
[9] A. U. Ernst, D. T. Bowers, L. H. Wang, K. Shariati, M. D. Plesser, N. K. Brown, T. Mehrabyan, M. Ma, Nanotechnology in cell replacement therapies for type 1 diabetes, Adv Drug Deliv Rev. 139 (2019) 116-138. https://doi.org/10.1016/J.ADDR.2019.01.013.
[10] C. C. Chang, E. D. Boland, S. K. Williams, J. B. Hoying, Direct-write bioprinting three-dimensional biohybrid systems for future regenerative therapies, J Biomed Mater Res B Appl Biomater. 98B (2011) 160-170. https://doi.org/10.1002/JBM.B.31831.
[11] X. Wang, K. G. Maxwell, K. Wang, D. T. Bowers, J. A. Flanders, W. Liu, L. H. Wang, Q. Liu, C. Liu, A. Naji, Y. Wang, B. Wang, J. Chen, A. U. Ernst, J. M. Melero-Martin, J. R. Millman, M. Ma, A nanofibrous encapsulation device for safe delivery of insulin-producing cells to treat type 1 diabetes, Sci Transl Med. 13 (2021). https://doi.org/10.1126/scitranslmed.abb4601.
[12] W. Liu, J. A. Flanders, L. H. Wang, Q. Liu, D. T. Bowers, K. Wang, A. Chiu, X. Wang, A. U. Ernst, K. Shariati, J. S. Caserto, B. Parker, D. Gao, M. D. Plesser, L. G. Grunnet, C. Rescan, R. Pimentel Carletto, L. Winkel, J. M. Melero-Martin, M. Ma, A Safe, Fibrosis-Mitigating, and Scalable Encapsulation Device Supports Long-Term Function of Insulin-Producing Cells, Small. 18 (2022). https://doi.org/10.1002/smll.202104899.
[13] S. Harrington, J. Williams, S. Rawal, K. Ramachandran, L. Stehno-Bittel, Hyaluronic Acid/Collagen Hydrogel as an Alternative to Alginate for Long-Term Immunoprotected Islet Transplantation*, Https://Home.Liebertpub.Com/Tea. 23 (2017) 1088-1099. https://doi.org/10.1089/TEN.TEA.2016.0477.
[14] K. K. Papas, H. de Leon, T. M. Suszynski, R. C. Johnson, Oxygenation strategies for encapsulated islet and beta cell transplants, Adv Drug Deliv Rev. 139 (2019) 139-156. https://doi.org/10.1016/J.ADDR.2019.05.002.
[15] D. T. Bowers, M. L. Tanes, A. Das, Y. Lin, N. A. Keane, R. A. Neal, M. E. Ogle, K. L. Brayman, C. L. Fraser, E. A. Botchwey, Spatiotemporal oxygen sensing using dual emissive boron dye-polylactide nanofibers, ACS Nano. 8 (2014) 12080-12091. https://doi.org/10.1021/NN504332J.
[16] I. Constantinidis, J. R. Robert Long, C. Weber, S. Safley, A. Sambanisb, Non-Invasive Monitoring of a Bioartificial Pancreas in Vitro and in Vivo, Ann N Y Acad Sci. 944 (2001) 83-95. https://doi.org/10.1111/J.1749-6632.2001.TB03825.X.
[17] L. Jansson, A. Barbu, B. Bodin, C. J. Drott, D. Espes, X. Gao, L. Grapensparr, Ö. Källskog, J. Lau, H. Liljebäck, F. Palm, M. Quach, M. Sandberg, V. Strömberg, S. Ullsten, P. O. Carlsson, Pancreatic islet blood flow and its measurement, Ups J Med Sci. 121 (2016) 81-95. https://doi.org/10.3109/03009734.2016.1164769.
[18] J. H. Jeong, V. Chan, C. Cha, P. Zorlutuna, C. Dyck, K. J. Hsia, R. Bashir, H. Kong, “Living” microvascular stamp for patterning of functional neovessels; Orchestrated control of matrix property and geometry, Advanced Materials. 24 (2012) 58-63. https://doi.org/10.1002/adma.201103207.
[19] W. Song, A. Chiu, L. H. Wang, R. E. Schwartz, B. Li, N. Bouklas, D. T. Bowers, D. An, S. H. Cheong, J. A. Flanders, Y. Pardo, Q. Liu, X. Wang, V. K. Lee, G. Dai, M. Ma, Engineering transferrable microvascular meshes for subcutaneous islet transplantation, Nat Commun. 10 (2019). https://doi.org/10.1038/s41467-019-12373-5.
[20] S. Song, C. Blaha, W. Moses, J. Park, N. Wright, J. Groszek, W. Fissell, S. Vartanian, A. M. Posselt, S. Roy, An intravascular bioartificial pancreas device (iBAP) with silicon nanopore membranes (SNM) for islet encapsulation under convective mass transport, Lab Chip. 17 (2017) 1778-1792. https://doi.org/10.1039/c71c00096k.
[21] R. Shaheen, R. E. Gurlin, R. Gologorsky, C. Blaha, P. Munnangi, A. Santandreu, A. Torres, P. Carnese, G. G. Nair, G. Szot, W. H. Fissell, M. Hebrok, S. Roy, Superporous agarose scaffolds for encapsulation of adult human islets and human <scp>stem-cell-derived</scp>β cells for intravascular bioartificial pancreas applications, J Biomed Mater Res A. (2021) jbm.a.37236. https://doi.org/10.1002/JBM.A.37236.
[22] E. X. Han, J. Wang, M. Kural, B. Jiang, K. L. Leiby, N. Chowdhury, G. Tellides, R. G. Kibbey, J. H. Lawson, L. E. Niklason, Development of a Bioartificial Vascular Pancreas, Https://Doi.Org/10.1177/20417314211027714. 12 (2021) 204173142110277. https://doi.org/10.1177/20417314211027714.
[23] G. Reach, P. Poussier, A. Sausse, R. Assan, M. Itoh, J. E. Gerich, Functional Evaluation of a Bioartificial Pancreas Using Isolated Islets Perifused with Blood Ultrafiltrate, Diabetes. 30 (1981) 296-301. https://doi.org/10.2337/DIAB.30.4.296.
[24] Sullivan S J, Maki T, Borland K M, Mahoney M D, Solomon B A, Muller T E, Monaco A P, Chick W L, Biohybrid artificial pancreas: long-term implantation studies in diabetic, pancreatectomized dogs, Science. 252 (1991) 718-721. https://doi.org/10.1126/SCIENCE.2024124.
[25] A. P. Monaco, T. Maki, H. Ozato, M. Carretta, S. J. Sullivan, K. M. Borland, M. D. Mahoney, W. L. Chick, T. E. Muller, J. Wolfrum, B. Solomon, Transplantation of islet allografts and xenografts in totally pancreatectomized diabetic dogs using the Hybrid Artificial Pancreas, in: Ann Surg, Lippincott, Williams, and Wilkins, 1991: pp. 339-362. https://doi.org/10.1097/00000658-199109000-00016.
[26] Maki T, Ubhi C S, Sanchez-Farpon H, Sullivan S J, Borland K, Muller T E, Solomon B A, Chick W L, Monaco A P, Successful treatment of diabetes with the biohybrid artificial pancreas in dogs, Transplantation. 51 (1991) 43-51. https://doi.org/10.1097/00007890-199101000-00006.
[27] L. R. Nyman, E. Ford, A. C. Powers, D. W. Piston, Glucose-dependent blood flow dynamics in murine pancreatic islets in vivo, Am J Physiol Endocrinol Metab. 298 (2010). https://doi.org/10.1152/AJPENDO.00715.2009.
[28] J. A. Diez, R. Arrojo e Drigo, X. Zheng, O. v. Stelmashenko, M. Chua, R. Rodriguez-Diaz, M. Fukuda, M. Köhler, I. Leibiger, S. B. B. Tun, Y. Ali, G. J. Augustine, V. A. Barathi, P. O. Berggren, Pancreatic Islet Blood Flow Dynamics in Primates, Cell Rep. 20 (2017) 1490-1501. https://doi.org/10.1016/J.CELREP.2017.07.039.
[29] N. J. Hogrebe, K. G. Maxwell, P. Augsornworawat, J. R. Millman, Generation of insulin-producing pancreatic f cells from multiple human stem cell lines, Nature Protocols 2021 16:9. 16 (2021) 4109-4143. https://doi.org/10.1038/s41596-021-00560-y.
[30] N. J. Hogrebe, P. Augsornworawat, K. G. Maxwell, L. Velazco-Cruz, J. R. Millman, Targeting the cytoskeleton to direct pancreatic differentiation of human pluripotent stem cells, Nat Biotechnol. 38 (2020) 460-470. https://doi.org/10.1038/s41587-020-0430-6.
[31] M. Yu, D. Agarwal, L. Korutla, C. L. May, W. Wang, N. N. Griffith, B. J. Hering, K. H. Kaestner, O. C. Velazquez, J. F. Markmann, P. Vallabhajosyula, C. Liu, A. Naji, Islet transplantation in the subcutaneous space achieves long-term euglycaemia in preclinical models of type 1 diabetes, Nature Metabolism 2020 2:10. 2 (2020) 1013-1020. https://doi.org/10.1038/S42255-020-0269-7.
[32] S. Tabakova, E. Nikolova, S. Radev, Carreau model for oscillatory blood flow in a tube, AIP Conf Proc. 1629 (2015) 336. https://doi.org/10.1063/1.4902290.

1-25. (canceled)

26. An intravascular artificial pancreas device capable of producing insulin comprising:
a first vascular layer comprising a plurality of first vascular channels each having a first end and a second end, wherein each of the first ends of the plurality of vascular channels connects to a first input conduit and each of the second ends of the plurality of vascular channels connects to a first output conduit, thereby forming a first vascular channel network;
an islet layer comprising pancreatic islet and/or beta cells disposed within at least one islet chamber, wherein the pancreatic islet and/or beta cells are further embedded within an islet chamber matrix; and
a first thin electrospun membrane disposed as a biomolecule and gas permeable interface between the plurality of vascular channels and a first side of the at least one islet chamber, the plurality of vascular channels and the first side of the at least one islet chamber juxtaposed from each other across the first thin electrospun membrane, thereby permitting exchange of biomolecules and gases between the pancreatic islet and/or beta cells disposed within the at least one islet chamber of the islet layer and the plurality of vascular channels in the first vascular layer.

27. The intravascular artificial pancreas of claim 26, wherein the at least one islet chamber comprises a plurality of islet chambers configured as a plurality of islet channels, and at least two vascular channels of the plurality of vascular channels interface with and are juxtaposed across from a first side of each islet channel through a first thin electrospun membrane.

28. The intravascular artificial pancreas device of claim 26, further comprising:
a second vascular layer comprising a plurality of vascular channels, each having a first end and a second end, wherein each of the first ends of the plurality of vascular channels connects to a second input conduit and each of the second ends of the plurality of vascular channels connects to a second output conduit, thereby forming a second vascular channel network; and
a second thin electrospun membrane disposed as a biomolecule and gas permeable interface between the plurality of vascular channels of the second vascular layer and a second side of the at least one islet chamber, thereby permitting exchange of biomolecules and gases between the pancreatic islet and/or beta cells disposed within the at least one islet chamber and the plurality of vascular channels in the second vascular layer, optionally, wherein the first and/or second side of the at least one islet chamber interface with the plurality of vascular channels of the first and/or second vascular layer through the first and/or second thin electrospun membrane at a distance of approximately 10-30 micrometers (μm).

29. The intravascular artificial pancreas device of claim 28, wherein the at least one islet chamber comprises a plurality of islet chambers configured as a plurality of islet channels, and wherein at least two vascular channels of the plurality of vascular channels of the second vascular layer interface with and are juxtaposed across from a second side of each islet channel through a second thin electrospun membrane.

30. The intravascular artificial pancreas device of claim 26, wherein the plurality of vascular channels of the first vascular layer and/or the second vascular layer are lined with endothelial cells.

31. The intravascular artificial pancreas device of claim 26, wherein the beta cells are hypo-immune (B2M−/−, CIITA−/−) and/or derived from induced pluripotent stem cells (iPSC).

32. The intravascular artificial pancreas device of claim 30, wherein the endothelial cells are glomerular microvascular endothelial cells or human umbilical vein endothelial cells.

33. The intravascular artificial pancreas of claim 26, wherein the intravascular artificial pancreas device is glucose responsive, producing an amount of insulin in proportion to an amount of glucose within the device.

34. The intravascular artificial pancreas device of claim 26, wherein the plurality of vascular channels of the first vascular and/or the second vascular layer are microchannels that form a first and/or second microvascular channel network.

35. The intravascular artificial pancreas device of claim 27, wherein the at least one islet chamber comprises elongated first and/or second sides to permit increased surface area for interfacing with one or more microvascular channels across the first and/or second thin electrospun membrane.

36. The intravascular artificial pancreas device of claim 26, wherein the amount of islets and/or beta cells present in the device comprises at least 500,000 islet equivalents.

37. The intravascular artificial pancreas device of claim 26, wherein the islet chamber matrix comprises an in-situ polymerized matrix, optionally wherein the islet chamber matrix comprises collagen.

38. The intravascular artificial pancreas device of claim 26, wherein the first and/or second thin electrospun membrane comprises polycaprolactone and gelatin.

39. The intravascular artificial pancreas device of claim 26, wherein the islet layer and/or the first and/or second vascular layers further comprise an encasement matrix which encases the first and/or second vascular channel network and the at least one islet chamber.

40. The intravascular artificial pancreas device of claim 26, wherein the first and second inlet conduits are fluidly connected to a first end of a fluid supply conduit and the first and second outlet conduits are fluidly connected to a first end of a fluid exit conduit, optionally wherein the fluid supply conduit and fluid exit conduit comprise second ends which are proximate to each other and/or are on the same side of the device.

41. The intravascular artificial pancreas device of claim 26, wherein the first vascular layer, the islet layer, the first thin electrospun membrane and optionally the second vascular layer and second thin electrospun membrane form a first device unit, and wherein the intravascular artificial pancreas device further comprises a second device unit comprising a first vascular layer, an islet layer, a thin electrospun membrane and optionally a second vascular layer and second thin electrospun membrane, and wherein the inlet and outlet conduits of the first device unit are in fluid connection with the respective inlet and outlet conduits of the second device unit.

42. A process for manufacturing an intravascular artificial pancreas, comprising the steps of:
(A) generating a first thin nanofibrous membrane by electrospinning a polymer containing solution;
(B) depositing a sacrificial substrate in the form of at least one islet chamber upon a first side of the thin nanofibrous membrane;
(C) depositing a plurality of sacrificial substrates in the form of at least a plurality of first vascular channels upon a second side of the thin nanofibrous membrane, wherein the sacrificial substrates in the form of first vascular channels each have a first end and a second end, wherein first ends are connected to a first input conduit and second ends are connected to a first outlet conduit;
(D) encasing at least the thin nanofibrous membrane having the sacrificial substrates of step (B) and (C) deposited thereon within an encasement matrix;
(E) removing the sacrificial substrates of steps (B) and (C) to provide a first vascular network and at least one islet chamber; and
(F) filling the at least one islet chamber with an islet chamber matrix comprising islets and/or beta cells capable of producing proinsulin peptide and/or glucagon.

43. The process of claim 42, further comprising:
(C)(i) generating a second thin nanofibrous membrane by electrospinning a polymer containing solution over the sacrificial substrate in the form of at least one islet chamber; and
(C)(ii) depositing a plurality of sacrificial substrates in the form of a plurality of second vascular channels upon a side of the second thin nanofibrous membrane opposite the side in contact with the sacrificial substrate in the form of at least one islet chamber, wherein the sacrificial substrates in the form of second vascular channels each have a first end and a second end, wherein the first ends are connected to a second inlet conduit and the second ends are connected to a second outlet conduit;
wherein step (D) comprises encasing the second nanofibrous membrane, sacrificial substrates, the second inlet conduit and the second outlet conduit of (C)(i)-(ii) together with the first thin nanofibrous membrane, sacrificial substrates, first inlet conduit, and first outlet conduit of (A)-(C) within an encasement matrix; and
wherein step (E) further comprises removing the sacrificial substrates of (C)(ii) to provide a second vascular network.

44. The process of claim 42, further comprising:
(G) introducing a suspension of endothelial cells into the first vascular network and/or the second vascular network, and after a period of time, flipping the device 180 degrees.

45. The process of claim 43, further comprising:
(C)(iii) generating a third thin nanofibrous membrane by electrospinning a polymer containing solution over the plurality of sacrificial substrates formed in step (C); and
wherein step (D) further comprises encasing the third nanofibrous membrane, second nanofibrous membrane, sacrificial substrates, the second inlet conduit and the second outlet conduit of (C)(i)-(ii) together with the first thin nanofibrous membrane, sacrificial substrates, first inlet conduit, and first outlet conduit of (A)-(C) within an encasement matrix.
